(19) United States
(12) Patent Application Publication
(10) Pub. No.: US 2025/0180456 A1
(43) Pub. Date: Jun. 5, 2025

(54) A METHOD AND SYSTEM FOR IDENTIFYING, SORTING AND COLLECTING ANALYTES, FOR EXAMPLE CELLS, IN A SAMPLE FLUID

(71) Applicant: TECHNISCHE UNIVERSITEIT EINDHOVEN, AE EINDHOVEN (NL)

(72) Inventors: Sertan Sukas, AE EINDHOVEN (NL); Jacob Marinus Jan Den Toonder, AE EINDHOVEN (NL)

(21) Appl. No.: 18/833,599

(22) PCT Filed: Jan. 31, 2023
(86) PCT No.: PCT/NL2023/050039

§ 371 Date: Jul. 26, 2024

(30) Foreign Application Priority Data
Jan. 31, 2022 (NL) 2030788

(51) Int. Cl. G01N 15/01; G01N 15/12; G01N 15/14; G01N 15/149; G01N 33/49

(52) U.S. Cl. CPC G01N 15/01; G01N 15/12; G01N 15/14; G01N 15/149; G01N 33/4915; G01N 2015/016

(57) ABSTRACT

A method for characterizing analytes, for example cells, in a sample fluid, is disclosed, the method comprising: passing a sample liquid containing analytes of at least a first type and analytes of a second type along a flow path through a fluid channel; analysing, in a first analysing unit including the fluid channel, the analytes, thereby obtaining first parameter values of at least one analyte characteristic associated with the analytes of the at least first type and second type, altering a further analyte characteristic associated with the analyte of the first type compared to the further analyte characteristic associated with the analyte of the second type; analysing, in a second analysing unit including the fluid channel, the analytes, thereby obtaining second parameter values of the at least one analyte characteristic associated with the analytes of the at least first type and second type; characterizing, in the processing unit, the analytes of the first type from the analytes of the second type by comparing the second parameter values with the first parameter values.

A method and system for identifying, sorting and collecting analytes, for example cells, in a sample fluid.

### TECHNICAL FIELD

The present disclosure relates to a method and a microfluidic system platform to be used to characterize (identify), enumerate, classify, sort and extract/collect targeted particle/cell populations in suspension, e.g., rare cells circulating bodily fluids, such as blood. Although the present disclosure primarily focuses on circulating tumour cells (CTCs), it can also be used for any analyte or rare cell population, such as circulating endothelial cells (CECs), circulating melanoma cells (CMCs), circulating hybrid cells (CHCs), etc.

### BACKGROUND OF THE DISCLOSURE

According to the WHO, cancer is the second leading cause of death worldwide, and caused around 10 million deaths in 2020. Globally, about 1 in 6 deaths is due to cancer. There were around 20 million new cancer cases globally in 2020 and this number is expected to reach around 30 million by 2040, causing 16 million deaths (Source: WHO IARC Globocan 2020).

Cancer is a deadly disease, however what kills is not the primary tumour but metastasis, which is spread of cancer. 90% of cancer related deaths are due to metastasis, therefore rapid identification of this process is critical for cancer treatment. CTCs are the most informant biomarkers for metastasis. They detach from the primary tumour and enter the bloodstream to eventually create a secondary tumour at another site. As they have the potential to stem for a new tumour formation in a separate tissue, their detection in blood is critical to assess the metastatic progression, to guide the therapy and to test and develop drugs. Early diagnosis of metastatic progression has uttermost importance to increase the survival rate and shorten the treatment period. In addition to diagnosis and prognosis, recurrence monitoring is also possible by detecting dormant CTCs in blood.

Real-time assessment of CTCs routinely in metastatic cancer patients in a non-invasive manner is crucial to assess the efficacy of therapy. Collection of CTCs is potentially possible via liquid biopsy, where a few mL of blood is drawn from the patient and traces of cancer are probed in the blood sample. Liquid biopsy is minimally invasive and brings many advantages over solid biopsy, where the tumour tissue is taken by surgery. However, retrieval of CTCs is a big challenge. In 1 mL of blood, there are billions of peripheral blood cells, where only 1-10 CTCs present. Their extreme rarity and inherent heterogeneity require sensitive, target specific and high throughput systems.

The present disclosure provides a technical solution for the above problem.

### SUMMARY OF THE DISCLOSURE

According to a first example of the disclosure, a method for characterizing analytes, for example cells, in a sample fluid is proposed, the method comprising passing a sample liquid containing analytes of at least a first type and analytes of a second type along a flow path through a fluid channel; analysing, in a first analysing unit including the fluid channel, the analytes of the at least first type and second type in the sample liquid, thereby obtaining first parameter values of at least one analyte characteristic associated with the analytes of the at least first type and second type; and storing, in a processing unit, the first parameter values of the at least one analyte characteristic.

An exemplary method further comprises altering a further analyte characteristic associated with the analyte of the first type compared to the further analyte characteristic associated with the analyte of the second type. The analytes of the at least first type and second type in the sample liquid may then be advantageously analysed in a second analysing unit including the fluid channel, thereby obtaining second parameter values of the at least one analyte characteristic associated with the analytes of the at least first type and second type.

An exemplary method further includes storing, in the processing unit, the second parameter values of the at least one analyte characteristic and characterizing, in the processing unit, the analytes of the first type from the analytes of the second type by comparing the second parameter values with the first parameter values.

This allows for an inline method capable of characterizing analytes from other analytes. In particular, very rare cells or analytes, mentioned in this application as analytes of a second type, may be herewith effectively identified from peripheral blood cells, mentioned in this application as analytes of a first type.

According to an example of the method of the disclosure altering the further analyte characteristic comprises the step of subjecting, in a device including the fluid channel, the analytes of the at least first type and second type in the sample liquid to an electric field.

In some embodiments, the method according to the disclosure, further comprises the step of separating in a separating unit mounted to the fluid channel, the analytes of the first type from the analytes of the second type based on the characterization in the processing unit and using the altered further analyte characteristic of the analyte of the first type. Accordingly, by separating the peripheral cells (analytes of the first type) from the sample fluid, the rare cells or analytes of the second type can also be efficiently separated and analysed for further research.

The step of separating the analytes of the first and second type may comprise applying an electric field to the sample fluid, and using the electric field to divert the analyte of the second type from the flow path. Additionally or alternatively, a valve can be used. The analyte of the second type can then be diverted from the flow path by guiding the flow to a desired outlet by changing the position of the valve. After separating the analytes of the first and second type, analytes of the second type may be collected in a collection reservoir.

Additionally, in a further example of the method according to the disclosure, it comprises the step of sorting the analytes of at least the first and second type on analyte size. This provides a prior selection of the analytes in the sample fluid further improving the efficiency and accuracy of the characterizing and separation steps of the method.

As the method according to the disclosure processes large amounts of data, the step of characterizing of the analytes of the first type from the analytes of the second type is preferably performed using one or more machine learning algorithms.

Additionally, one or more analysing steps of the method according to some embodiments of the present disclosure comprise electrical impedance spectroscopy (EIS), in particular multi-frequency electrical impedance spectroscopy.

According to an example of the present disclosure, the step of altering a further analyte characteristic comprises the step of altering a membrane or causing a mechanical cell deformation of an analyte and wherein the further analyte characteristic is the electrical impedance response of the analyte. In particular, if the altering step comprises charging outer membrane and/or electroporation, and the further analyte characteristic is the electrical properties of the analyte membrane, one of the analyte types can be effectively tagged for characterizing, in particular identification.

Methods according to the present disclosure may advantageously include the step of optically sensing a sensing region of the fluid channel.

According to another embodiment of the disclosure, a method for characterizing analytes, for example cells, in a sample fluid, is proposed that comprises performing the steps of any one or more of the foregoing methods according to the present disclosure, concurrently, in a plurality of different characterizing lines. The plurality of characterizing lines may receive analytes that have been sorted, for example, by size, during the initial step of sorting the analytes in the sample fluid to different of the plurality of the characterizing lines.

An example of a system for characterizing analytes, for example cells, in a sample fluid according to the disclosure includes at least one characterizing line, which line comprises at least a fluid channel defining a flow path having an inlet and an outlet and structured to allow a sample liquid containing analytes of at least a first type and analytes of a second type to pass through along the flow path. Between the inlet and the outlet seen in the direction of the flow path several units can be implemented, which perform process steps on the sample fluid, in particular on the analytes of the at least first and second type.

A first exemplary analysing unit includes the fluid channel and is structured to analyse the analytes of the at least first type and second type in the sample liquid and structured to obtain first parameter values of at least one analyte characteristic associated with the analytes of the at least first type and second type.

An altering unit also includes the fluid channel and may be located downstream from the first analysing unit structured for altering a further analyte characteristic associated with the analyte of the first type compared to the further analyte characteristic associated with the analyte of the second type. In an embodiment, the altering unit comprises an electric field generating unit, structured to subject the analytes of the at least first type and second type in the sample liquid to an electric field, thereby altering the further analyte characteristic associated with the analyte of the first type compared to the further analyte characteristic associated with the analyte of the second type. In particular, the altering unit may be structured for altering a membrane or causing a mechanical cell deformation of an analyte and wherein the further analyte characteristic is an electrical impedance response.

A second analysing unit including the fluid channel may be provided downstream from the altering unit. The second analysing unit is structured to analyse the analytes of the at least first type and second type in the sample liquid and is structured to obtain second parameter values of the at least one analyte characteristic associated with the analytes of the at least first type and second type.

The large amounts of data are processed by a processing unit, which is structured to acquire and store the first parameter values and second parameter values from the first and second analysing unit and to characterize the analytes of the first type from the analytes of the second type by comparing the second parameter values with the first parameter values.

This allows for an inline method capable of characterizing analytes from other analytes. In particular, very rare cells or analytes, mentioned in this application as analytes of a second type, are herewith effectively identified from peripheral blood cells, mentioned in this application as analytes of a first type.

Additionally, in an example of the system according to the disclosure, it comprises a separating unit including the fluid channel downstream from the second analysing unit. The separating unit is structured to separate the analytes of the first type from the analytes of the second type using the altered further analyte characteristic of the analyte of the first type, based on characterization signals generated and output by the processing unit.

By separating the analytes of the first type from the sample fluid, the analytes of the second type can also be efficiently separated and collected. For instance, the separating unit can comprise an electric field generating unit structured to apply an electric field to the sample fluid when it receives an activation signal from the processing unit for diverting the analyte of the second type from the flow path for its collection. Accordingly, exemplary embodiments of the system may include one or more collection unts, e.g., for collecting analytes of the second type. Some exemplary separating units may comprise a valve unit structured to divert the analyte of the second type from the flow path.

To further improve the efficiency and accuracy of the characterizing and separation of the several types of analytes, a prior sorting of the analytes in the sample fluid is obtained by implementing a sorting unit, which is including the fluid channel upstream from the first analysing unit and which is structured to sort the analytes of at least the first and second type on analyte size without making any selection.

An improved processing of the large amounts of data is achieved, as in a further beneficial example, the processing unit implements one or more machine learning algorithms for characterizing the analytes of the first type from the analytes of the second type.

Preferably, the first and second analysing unit comprise electrical impedance spectroscopy (EIS) means, in particular multi-frequency electrical impedance spectroscopy means, and furthermore the microfluidic device can be structured to charge and/or electroporate the membrane of the analyte of the first type.

Exemplary embodiments of the present disclosure may also include an optic sensing unit structured to optically sense a sensing region of the fluid channel.

Another embodiment of the present disclosure is a platform for characterizing analytes, for example cells, in a sample fluid, concurrently in a plurality of systems according to embodiments of the present disclosure. The platform comprises a plurality of characterizing lines associated with a plurality of systems, each system structured for characterizing analytes, for example cells, in a sample fluid. Such exemplary embodiments may further include a sorting device disposed upstream of the plurality of the systems and structured to sort analytes in the sample fluid to different characterizing lines of the plurality of systems.

### BRIEF DESCRIPTION OF THE DRAWINGS

The invention will now be discussed with reference to the drawings, which show in:

FIG. 1 the workflow of the platform or system according to the disclosure;

FIG. 2 working principle of the platform or system according to the disclosure;

FIG. 3 illustrates the physical parameters that are measured for multi-frequency and multi-parameter electrical impedance measurement of cells for tumour cell discrimination;

FIG. 4 depicts raw data for multifrequency measurements of tumour cell, white blood cell and a cluster of tumour and white blood cells.

FIG. 5 describes the method according to the disclosure for combined electrical detection and alteration for tagging peripheral blood cells for maximizing detection specificity;

FIG. 6 depicts schematically an example of a microfluidic device according to the disclosure as well as illustration of measurement methodology according to the disclosure;

FIGS. 7a and 7b depict schematically other examples of microfluidic devices according to the disclosure;

FIGS. 8a and 8b illustrate the examples of downstream separating unit.

### DETAILED DESCRIPTION OF THE DISCLOSURE

For a proper understanding of the disclosure, in the detailed description below corresponding elements or parts of the disclosure will be denoted with identical reference numerals in the drawings.

The technology according to the disclosure is designed for tumour cell detection, enumeration, purification, and collection. The design is formed of a versatile micro-electro-fluidic system (MEFS) that is based on fast and sensitive electrical impedance measurements with the option of coupling to optics for expanding the capabilities, such as real-time characterization. A technique and device for real-time characterization is described in applicant's other PCT application, filed concurrently with the present application, which other PCT application claims priority to the Dutch application no. 2030787 titled “A microfluidic device for detecting and characterizing at least one analyte, for example a cell, in a sample fluid.” filed on 31 Jan. 2022, the disclosure of which is incorporated by reference herein.

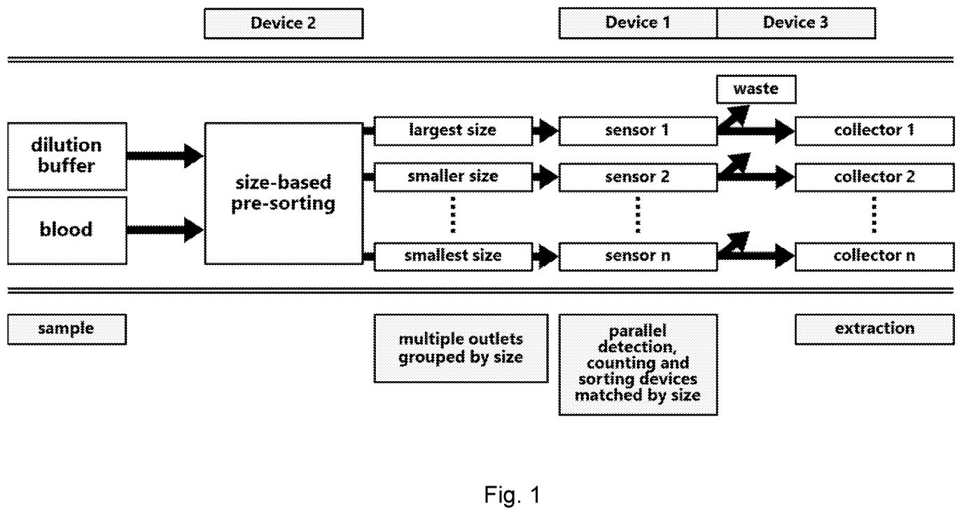

FIG. 1 describes the system level design for an example of the platform according to the disclosure. On-chip dilution is applied to blood sample prior to size-based pre-sorting. This initial sorting step performed in the sorting device 2 is not for exclusion but for sorting based on size as well as for parallelization. Output of the sorting device 2 includes a plurality of fluid channels containing analytes that have been sorted by size, designated as largest size, smaller size, . . . , smallest size. The cells (in general analytes) of the one or more, and preferably plurality, of output fluid channels of the sorting device 2 are sent to detection or analysing units that are working in parallel based on size distribution. In an exemplary embodiment, one or more fluid channels containing analytes that have been sorted by size, designated as largest size, smaller size, . . . , smallest size, are directed to different modular parts of the device 1, such as sensor 1, sensor 2, . . . , sensor n. There, the analytes can be detected, counted and/or sorted in parallel. The dimensions of each analysing unit of different modular parts, such as sensor 1, sensor 2, . . . , sensor n, are adjusted to size the measurement volume for matching with the analyte size, resulting in a further increase in the sensitivity and avoiding simultaneous detection of multiple (overlapping) events.

After their identification, preferably concurrently, in two or more of sensor 1, . . . , sensor n, analytes of a second type, such as rare cells, are separated from sensor 2, analytes of the first type, in the two or more fluid channels 1, 2, . . . , n, and steered to the two or more collection reservoirs 1, 2, . . . , n. Analytes of the first type, such as peripheral blood cells, are sent to waste.

FIG. 1 depicts in particular the workflow of the system (platform) according to an embodiment of the disclosure. The platform is designed to be a modular system with three devices that can be separated by tubing connections or fully integrated. The modularity is for having the platform ready for industrialization as failure of one device will not force one to replace the whole platform, but only the faulty device. Further design developments on any device does not affect the design of other devices. This can be considered an advantage for product development.

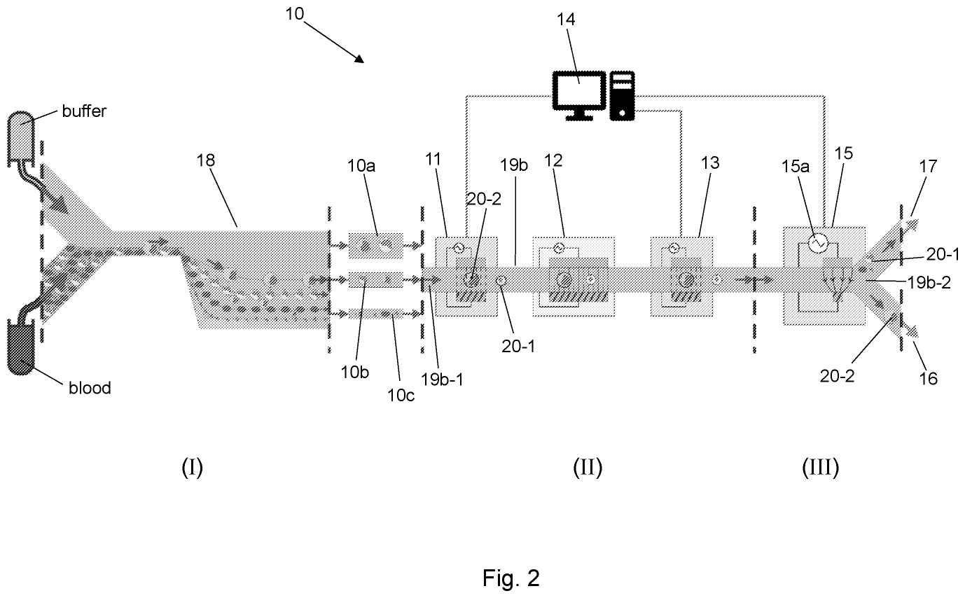

FIG. 2 illustrates how an exemplary unit or device works within the entire system according to the disclosure, the system being denoted with reference numeral 10. After size-based pre-sorting of the analytes in sample fluid or cell stream (stage I depicting a sorting unit 18 in FIG. 2), cells in one or more, and preferably two or more, branches or characterizing lines 10a-10b-10c are processed, preferably concurrently, through detection and enumeration unit(s), exemplified by the detection and enumeration unit 11 (first analysing unit: stage II). For simplicity, only one branch/characterizing line 10b is depicted in more detail in FIG. 2. Other branches/characterizing lines may have the same or similar construction and are preferably comprise components that are structured so that the measurement volume is sized according to the analyte size.

The exemplary branch/characterizing line 10b is composed around a fluid channel 19b defining a flow path having an inlet 19b-1 and an outlet 19b-2. The fluid channel 19b structured to allow a sample liquid containing analytes of at least a first type 20-1 and analytes of a second type 20-2 to pass from the sorting unit 18 along the flow path between the inlet 19b-1 towards the outlet 19b-2. Illustrations of the different cell types in channel branches 10a, 10b, and 10c are representative, analytes of any type can be sorted into 10a or 10b or 10c based on their size. Analytes of first or second or any type may fall into the same size range and then co-exist in the same channel branch (10a, 10b or 10c). Therefore, the operation of the size-based pre-sorting unit 18 is independent of the analyte type, but is dependent on the analyte size.

There are three subunits 11, 12, and 13 that are being operated sequentially within one exemplary embodiment of the system: sub-unit 11 (first analysing unit) includes a detector or sensor unit and serves to detect a characteristic of one or more analytes, such as the size, membrane properties, cytoplasm properties, nucleus properties, and genomic content or nucleic acids and sending the information to the processing unit 14. Sub-unit 12 (an alteration unit) is structured to alter a characteristic of an analyte, such as the membrane, of WBCs, for example, by charging or electroporating the outer (plasma) membrane of WBCs, while leaving the outer (plasma) membrane of CTCs intact. Sub-unit 13 (second analysing unit) includes a detector or a sensor unit, which may be the same as sub-unit 11, and serves to detect a characteristic of one or more analytes, such as the same properties as sub-unit 11, and identify the shift in the signal due to an altered analyte characteristic, e.g., for WBCs caused by altered membrane, in the sub-unit 12. Sub-unit 13 subsequently sends the signal to the processing unit 14 for tagging WBCs at sub-unit 13. The data that detection and enumeration unit generates are processed by the processing unit 14 using machine learning algorithms for identification of each cell type (analyte of at least the first and second type) and this information is used to activate the valve 15a in the purification device 15 (sorting unit 15) to sort and collect tumour cells (analytes of the second type 20-2) in the collection chamber (reservoir) 16 or discard the rest of the cells (analytes of the first type 20-1) via waste outlet 17 (stage III).

The working principle of the method and system according to the disclosure as outlined in FIG. 2, employs a “sample in result out” approach. This system of the present disclosure not only isolates and collects tumour cells (analytes of the second type) from peripheral blood cells (analytes of the first type 20-1) but also identify and enumerate them on the fly. Therefore, the composition of the output (collected analytes 20-2) of the system is already known.

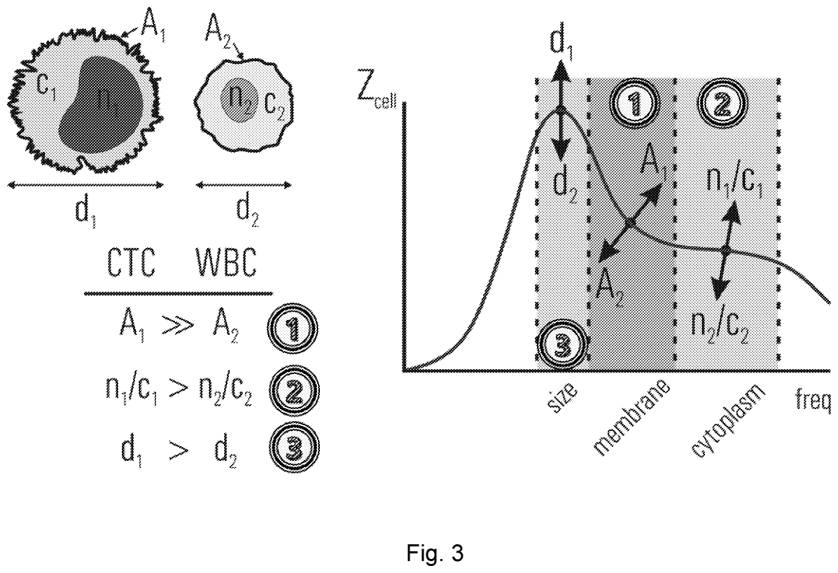

The proposed system aims to detect and combine the known physical differences between tumour cells (e.g. denoted as analytes of the second type 20-2) and peripheral blood cells (accordingly denoted as analytes of the first type 20-1). FIG. 3 illustrates some non-limiting examples of several of the physical features that have been commonly used for discriminating the tumour cells. In particular, FIG. 3 illustrates the physical parameters (first parameter values of at least one analyte characteristic) that are measured using multi-frequency and multi-parameter electrical impedance measurement of cells for tumour cell discrimination. A first property (a first analyte characteristic) being measured is the specific area A of the plasma membrane. The specific area A is denoted as total plasma membrane area normalized by the perimeter of the cell. For the same size, the specific area A₁ of the plasma membrane of a tumour cell typically is much higher compared to the specific membrane area A₂ of a white blood cell, hence A₁>>A₂. This difference can be measured for a certain frequency range as shown in the graph (section (1)) on the right of FIG. 3.

The second property (a second analyte characteristic) is the cytoplasmic content, such as the nuclear-to-cytoplasmic (N/C) ratio, cytoplasmic conductivity, cytoplasm composition or genomic content. Tumour cells typically represent higher N/C ratio (n₁/c₁) compared to white blood cells (n₂/c₂). This property can be derived by measuring the cells at a certain frequency range (section (2) in the graph on the right of FIG. 3). The third property (a third analyte characteristic) is the size (diameter) d, which can be used for identification and/or for normalizing (opacity calculation) the data obtained for the first and second properties. This is to increase the sensitivity and specificity of the measurements as any disturbance in flow velocity or a slight change in the position of the cell in the measurement volume may affect the signal. Normalization clears those effects from the collected signal and increases the signal quality.

To further increase the specificity of the measurements, the membrane of white blood cells is altered, yielding a shift in its electrical impedance response, this is the further analyte characteristic used for the tagging or characterizing steps. This allows tagging white blood cells for their accurate identification.

Typically before any liquid biopsy application, blood sample is diluted in red blood cell (RBC) lysis buffer for eliminating RBCs from the solution to increase the signal-to-noise ratio as RBCs outnumber any other cell population in blood. This process also affects the (electrical) properties of the outer membrane of white blood cells, while their viability is preserved.

RBC lysis buffer alters the properties of the membrane of white blood cells by causing reversible pore formation (as in electroporation) on the membrane of white blood cells, hence creating a shift in its electrical response. Therefore, immersing in RBC lysis buffer can be used as an alternative to electrical charging or electroporation of cell membranes.

As an alternative to electroporation or immersing in RBC lysis buffer, mechanical alternation of white blood cells can also be performed. As an example, cells can be flown through converging-diverging channels for mechanical alteration, such as nucleus relocation, which causes a shift in electrical response, too. Difference in deformability levels between tumour cells and white blood cells results in a differences in their electrical impedance response.

The change in the electrical response of white blood cells is measured after being immersed in a RBC lysis buffer. After the measurement it was validated that tumour cells remain intact and their electrical impedance response does not change.

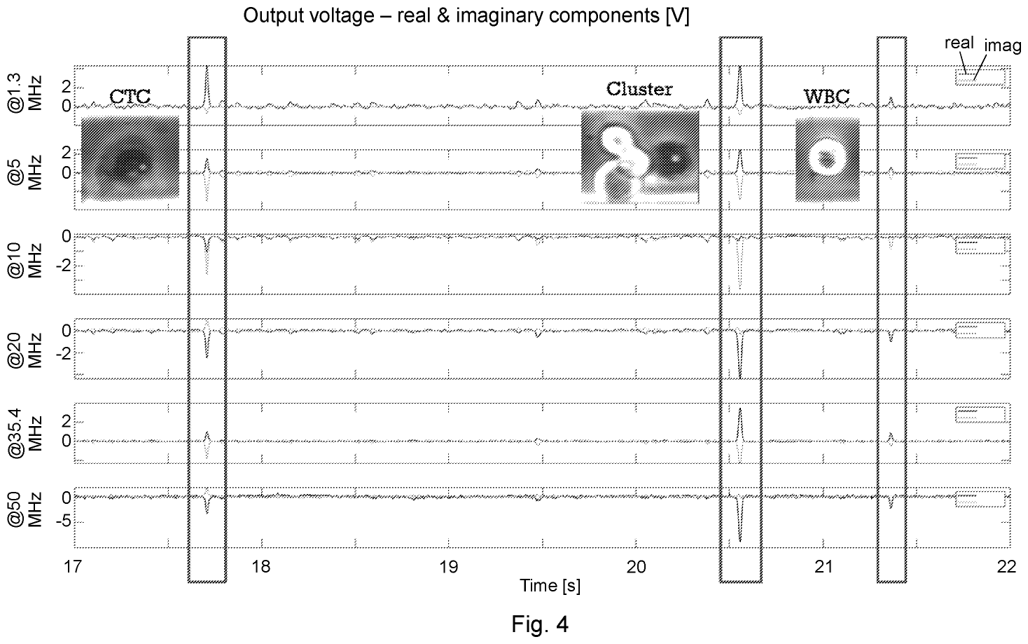

FIG. 4 depicts a typical data set that was collected as a proof on how electrical impedance measurements (the further analyte characteristic) clearly show this difference and it is possible to tag white blood cells. In particular, the raw data pertain to multifrequency measurements of tumour cell, white blood cell and a cluster (tumour cells and white blood cells). Data for frequencies at 10 MHz and 20 MHz reveal the different electrical responses for tagging. Processing the whole data set using machine learning algorithms provides enough information for discriminating or characterizing the different types of cells (analytes of the first and second type).

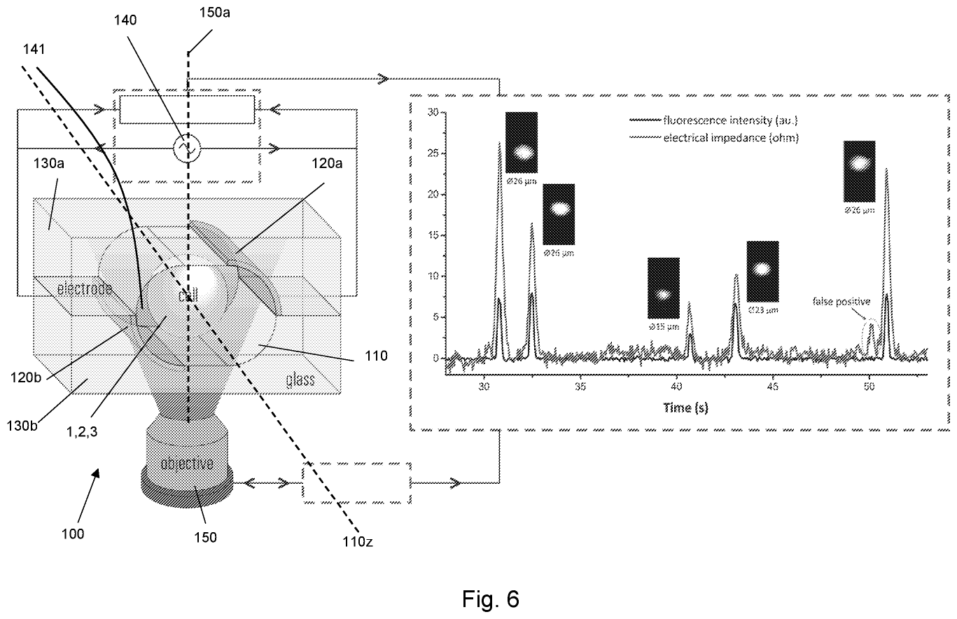

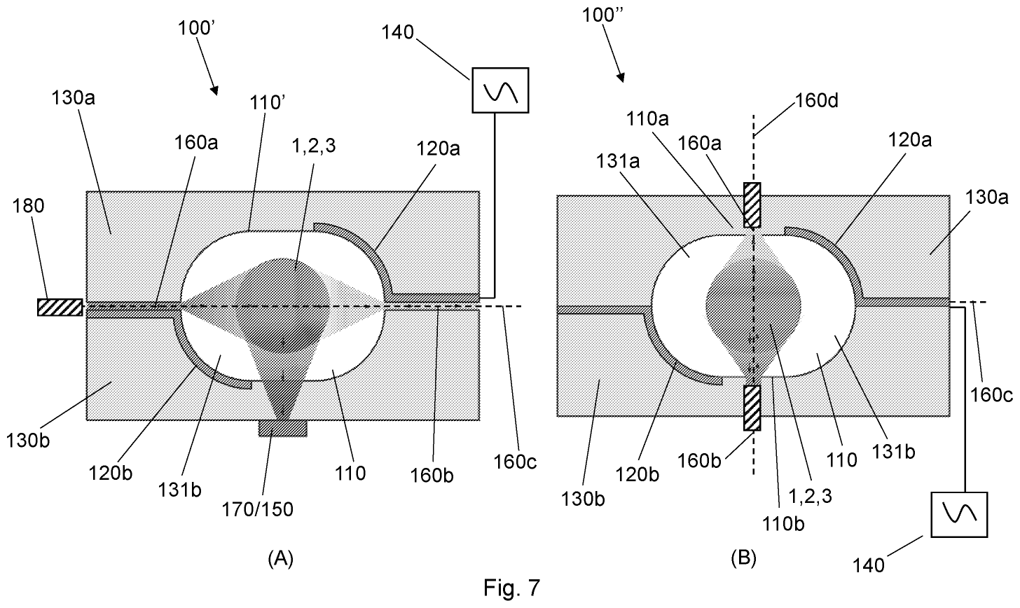

To have a better control over the process and to further increase the specificity, an electrical alteration of the outer (plasma) membrane of the white blood cells can be applied. Since the system according to the disclosure may be equipped with 3D electrodes, for example, those described in connection with and illustrated in FIGS. 6, 7a and 7b, for creating electric fields with high precision in terms of magnitude and homogeneity, it is possible to perform charging of or reversible electroporation on white blood cells while tumour cells remain intact as in the chemical case (immersing in RBC lysis solution).

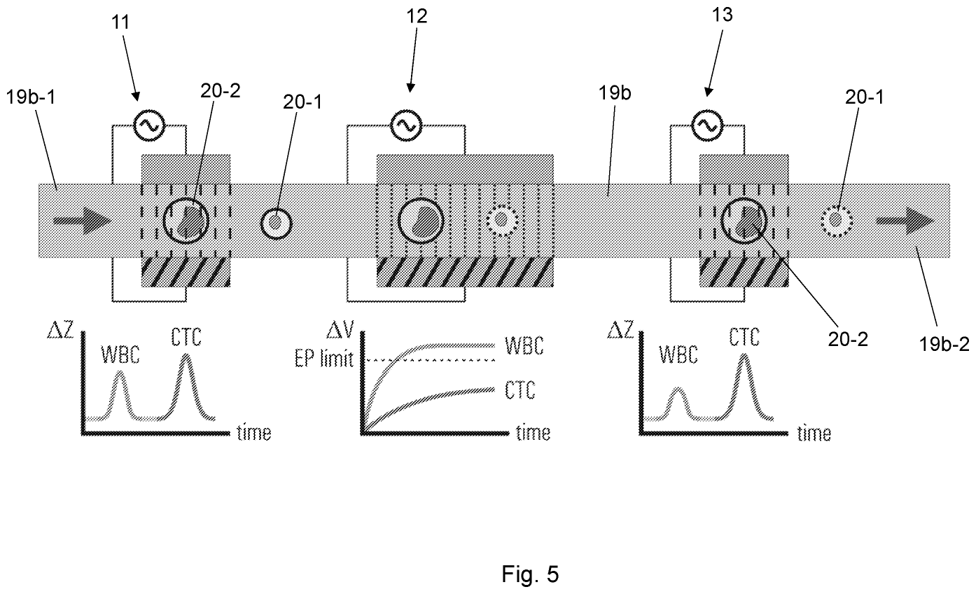

FIG. 5 further illustrates the steps for the combined detection and alteration of cells. In particular, FIG. 5 describes a method according to the disclosure for combined electrical detection and alteration for tagging white blood cells for further increasing the detection specificity. The electric field for charging the cell membranes and/or for electroporation (e.g., step 2 of FIG. 5 using the microfluidic device 12 in FIG. 2) is kept below the electroporation limit of tumour cells, hence, it does not alter their membrane properties (the further analyte characteristic), where it goes beyond the electroporation limit for white blood cells and eventually porates them. This poration process is reversible. In other words, the membrane heals fast and pores are closed. Comparing the signals from method step 1 for sensing the intact cells (analytes of the first and the second type) and method step 3 for sensing the intact (analytes of the second type) and treated (analytes of the first type) cells gives the possibility for identifying the difference in the electrical impedance response for the treated cells and is used for discrimination or characterization.

Being able to perform electroporation of cells with high level of control allows other applications, such as enhanced (content/drug) delivery into cells/organisms. One striking example would be the controlled electroporation of cells for genomic delivery for bioproduction. Without the requirement for purification after the treatment, this technology would replace the expensive chemical techniques, which also produce by-products, where e.g. bioburden becomes an issue and purification becomes a necessity. Moreover, online monitoring of the efficiency of delivery and the process of replication of nucleic acids is possible by coupling electrical sensing to electroporation. This can be applied to any cell or organism with an outer membrane (e.g., mammalian cells) and/or a wall (e.g., bacteria, yeast cells).

The disclosure allows for the analysis at single cell level and prevents that any cell (analyte) from being discarded or sent to the waste before being fully inspected. Presently known prior art systems provide only blind separation routines based on a specific target property by processing bulk volumes. Different from the existing solutions, this disclosure implements a sensor unit instead of a blind partitioning approach and enables an active decision making approach by this sensor unit for the collection of tumour cells or any target group/analytes within the sample. The detection and isolation of tumour cells as outlined in this disclosure are optionally label-free, which allow further downstream analysis, such as assessment of drug response for guiding the treatment or molecular analysis for drug development as the cells remain intact. The platform according to the disclosure will allow the utilization of such analyses on-the-go and in real-time with the coupling optical sensors or utilizing integrated optics into the device, for example, as described with reference to FIGS. 6, 7a, and 7b.

With the use of artificial intelligence (AI) and/or machine/deep learning techniques, the decision making as to the detection and isolation of tumour cells can already be implemented at the device level by integrating microprocessor for replacing a computer. Accordingly, the device platform and method according to the disclosure are versatile as the sensors being used can be tuned, activated, or deactivated depending on the desired application mode.

The system only requires dilution of blood samples as sample preparation. Therefore, total analysis times will be very short, and the costs will be low compared to most of the state-of-the-art systems that provide many antibody-based and/or multi-step approaches.

To further expand the capabilities of the system (e.g. on-the-fly characterization of cells), optical detection can be combined. One example of the disclosure comprises an optic sensing unit structured to optically sense a sensing area of the fluid channel, preferably, between the at least one pair of electrodes. This enables a system capable of electrical and optical detection of analytes, such as particles or cells in an electrolyte solution. For example, a full spectrum optical spectrometer and a high-speed camera as well as integrated optical waveguides can be used for collecting the optical data for label-free and/or fluorescent measurements. Fast optical data via the spectrometer is provided and on-demand imaging can be performed via high-speed CMOS cameras for visualization of the events of interest for end user.

FIG. 6 illustrates exemplary configurations of microfluidic devices that can be employed in one or more exemplary analysing units and/or altering units, e.g., 11, 12, 13. Some exemplary embodiments also incorporate an optic sensing unit. The microfluidic device 100 according to the disclosure allows for multifrequency electrical impedance measurements to be performed on analytes 1, 2, 3 (such as blood cells) passing through a fluid channel 110 having a longitudinal channel axis 110z using a pair of three-dimensional curved first and second electrodes 120a-120b, which are positioned about the longitudinal channel axis 110z. As shown in the Figures the first and second electrodes 120a-120b have a three-dimensional structured electrode surface positioned about the longitudinal channel axis 110z, thereby at least partly enveloping the fluid channel 110.

The microfluidic device 100 may include a first substrate 130a and a second substrate 130b. Materials suitable for making the one or more substrates 130a-130b include transparent materials, which are preferably compatible with microfabrication and thin-film processing techniques. Examples may include glass and silica. At least a portion of a wall of the fluid channel 110 may be formed by a first recess 131a in the first substrate 130a. Additionally or alternatively, at least a portion of the wall of the fluid channel 110 may be formed by a second recess 131b in the second substrate 130b. Preferably, the first recess 131a has the same shape as the second recess 131b, which greatly simplifies the manufacturing of the microfluidic devices. Most preferably, the first and second recesses 131a-131b are symmetric with respect to the mid- or central plane 160c (shown in FIG. 7) of the fluid channel 110.

The electrodes 120a-120b can be placed facing each other at different, preferably opposite, sides of the fluid channel 110. The first electrode 120a may be placed in the recess 131a of the first substrate 130a, and the second electrode 120b may be placed in the recess 131b of the second substrate 130b. Alternatively, one or more three-dimensional curved electrodes of the present disclosure, such as the electrodes 120a-120b, may be placed at another chosen location, such that the electrodes face each other and otherwise in accordance with the principles of the present disclosure. Examples of the processes that can be used to place the electrodes include, without limitation, evaporation, sputtering, or direct printing of metals. In the present disclosure, the term “placed” shall be interpreted to include within its meaning any type of depositing, arranging, mounting, integrating, or otherwise providing the one or more electrodes in any other manner consistent with the present disclosure.

Reference numeral 140 schematically depicts a known configuration of an electric field generating unit, which is connected with both electrodes 120a-120b and which is capable of applying an electric field distribution in the channel 110 between both electrodes 120a-120b. The electric field distribution in the fluid channel 110 between both electrodes 120a-120b is shown by the schematic electric field lines 141.

The microfluidic device 100 according to the disclosure may further include an optic sensing unit 150 arranged to optically detect or sense a region of the fluid channel 110 between the at least one pair of the first and second electrodes 120a and 120b. The optic sensing/detecting unit 150 can be structured as or include an optical waveguide, a laser device, and/or an optical sensor. In another beneficial example, the optical sensor can be a charge-coupled device (CCD), CMOS device, etc. In an embodiment of the present disclosure, optical measurements using an optic sensing unit 150 are performed concurrently, and preferably synchronously, with the electrical measurements, e.g., using electrodes 120a and 120b. Since the electrodes 120a-120b are disposed over only a portion of an inner surface wall 110′ of the fluid channel 110, they do not obstruct or hinder the optical sensing axis 150a of the optic sensing unit 150. Therefore, label-free (LED illumination, multi-focus or multi-depth arrangement, transmitted or reflected light, lens-free imaging, waveguide integration, PMT integration, etc.) or fluorescent (using specific biomarkers for staining) measurements are possible. In one exemplary microfluidic device 100, the optical sensing axis 150a may be perpendicular to the flow direction through the fluid channel 110, but in other exemplary embodiments it can be horizontal. In general, the optical sensing axis 150a may form any angle with the direction of the flow through the fluid channel 110 that allows for optical measurements to be made through the portion(s) of the first and or second substrate(s) 130a and 130b that are not obstructed by the electrode(s) 120a and 120b.

FIGS. 7a and 7b further illustrate exemplary configurations of microfluidic devices that can be employed in one or more exemplary analysing units and/or altering units, e.g., 11, 12, 13. These exemplary embodiments may also incorporate an optic sensing unit.

FIG. 7a, depicting example 100′, illustrates an exemplary embodiment of the integrated optics utilizing waveguide units 160a and 160b. One or more waveguide units, e.g., 160a-160b, may be disposed at or in the intermediate region between the substrates 130a and 130b, designated schematically by the mid- or center plane 160c. In this exemplary embodiment, the waveguide units 160a-160b are disposed between the first substrate 130a and the second substrate 130b, preferably, where substrates 130a and 130b are attached or bonded to each other. The material for the waveguides is selected based on its required difference in refractive index compared to the substrate material relating to the intended application. This difference defines how the light is confined and guided. Waveguide units suitable for use in embodiments of the present disclosure can be created, for example, by depositing/coating the material (e.g., silica or polymeric material) and patterning it on the substrates 130a and 130b using microfabrication techniques, which are similar to the ones used for manufacturing the substrates. As an alternative to an integrated waveguide unit or units, a focused laser beam from a laser device 180 can be aligned with the plane 160c as an external component serving for the same purpose resulting in a lower performance, but simpler and cheaper manufacturing.

FIG. 7b illustrates an alternative configuration, denoted with reference numeral 100″, where the waveguide units 160a and 160b may be disposed at the bottom surface 110b and the top surface 110a of the channel 110, respectively. Waveguide units may be disposed at or in the substrates 130a and 130b. In one embodiment, the optical axis of one or more waveguide units 160a and 160b is disposed along a plane 160d. The plane 160d is oriented at a non-zero angle, and, preferably, generally orthogonally with respect to the mid- or central plane 160c. This configuration requires the waveguides to be integrated after forming the recesses 131a and 131b on the substrates 130a and 130b.

The manufacturing process and material options can be the same as those described in connection with the previous configuration, shown in FIG. 7a. The material can be deposited onto a template or deposited and then patterned to form the waveguide, which can be disposed on the surface of the fluid channel 110 as the thickness of the waveguide is around two orders of magnitude smaller than the depth of the fluid channel 110. Therefore, the flow path is not disturbed by the waveguide. For shallow channels, where the thickness of the waveguide causes disturbance in the flow field, a recess can be created by removing material from the substrate to obtain a leveled surface for the channel 110 after disposing the waveguides.

As an alternative to integrated waveguides, far field optics can be used for this configuration. The optical components, such as light sources focusing the light and image sensors for collecting and sensing the scattered light can be placed at the top and bottom of the device externally (outside). The waveguide units 160a and 160b of both examples 100′ and 100″ of FIGS. 7a and 7b, are optically coupled to a sensing region of the fluid channel between at least two electrodes 120a and 120b to provide additional integrated light paths enabling illumination of the analytes 1, 2, 3 (particles or cells) with a (focused) light source and collecting the scattered light in different planes with several angles optionally in combination with a detector 170 for analyzing the analytes. The detector 170 may be located off-set from the light path 160c or 160d of the waveguide units 160a-160b. In such embodiments of the present disclosure, the optical axis of the detector forms a non-zero angle with the optical axis of the waveguide. Using external optical components as an alternative or in combination with integrated waveguides can add more functionality to the system and decrease the total cost.

In a first example, illustrated in FIG. 7a, light exiting a waveguide 160a or a focused (laser) beam 180 placed in the plane 160c of the channel 110 illuminating the analytes (cells or particles) 1, 2, 3 creates forward (FSC) and side (SSC) scatter, which can be read, detected, or sensed with an optic sensing/detecting unit 150, including a waveguide 160b and a detector 170, respectively. The detector 170 may be located off-set from the light path 160c of the waveguide units 160a and 160b. The detector 170 may be directly attached to the device 100′-100″ or placed with a distance based on the requirements of optical sensing application or arrangement of the optical setup or peripheral components.

In a second example, as illustrated in FIG. 7b, the waveguide units 160a and 160b are integrated at the top plane 110a and/or bottom plane 110b of the channel 110 for illumination and collecting forward and back scattered light. Waveguide unit 160a illuminates the analytes 1, 2, 3 and collects back scattered light, while waveguide unit 160b collects the forward scattered light. Second option is more suitable for shallow fluid channels 110. In some embodiments of the present disclosure, the first wave guide unit 160a and/or a second waveguide unit 160b are integrated at a first substrate 130a and/or a second substrate 130b.

Exemplary microfluidic devices described above may be advantageously included in exemplary altering units to perform charging of or reversible electroporation of subject analytes. Thus, in addition to being used as a sensor, some embodiments of the microfluidic device can be used for altering, such as electrical treatment and manipulation of analytes, such as cells and particles in suspension. The ability of controlling the electrode area that is in contact with the electrolyte by defining the coverage angle of the concave electrodes provides high level of control on the parameters that are applied for electrical treatment and manipulation. Alteration or treatment of particles or cells can be but is not limited to electroporation of cells for content delivery or extraction of inner cell content after e.g., bioproduction. Pore formation during electroporation can be reversible or irreversible as this process (electrode area, applied voltage, etc.) is well controlled and efficient with the proposed configuration. Alteration or manipulation of particles or cells can be but is not limited to guiding them by creating dielectrophoretic forces on these particles/cells. These forces can be generated by targeting a specific property, such as size, morphology, or phenotype, and by adjusting the frequency and the amplitude of the excitation voltage based on the selected property. Dielectrophoretic force then can be used to steer or direct the particles or cells in flow, or collect/concentrate them on defined areas in the device for follow-up downstream analyses.

The additional optical data obtained with the optic sensing unit provide information on physical properties, such as inner and outer morphology, size, shape, and phenotype. By combining these electric and optical data sets, the systems and methods according to the disclosure can be used as a flow cytometer for detection, enumeration, characterization, and/or classification of target analytes or target particles in a suspension, such as biological cells in blood. The systems and methods according to the disclosure can also be used as a hematology analyzer for blood cell classification and counting.

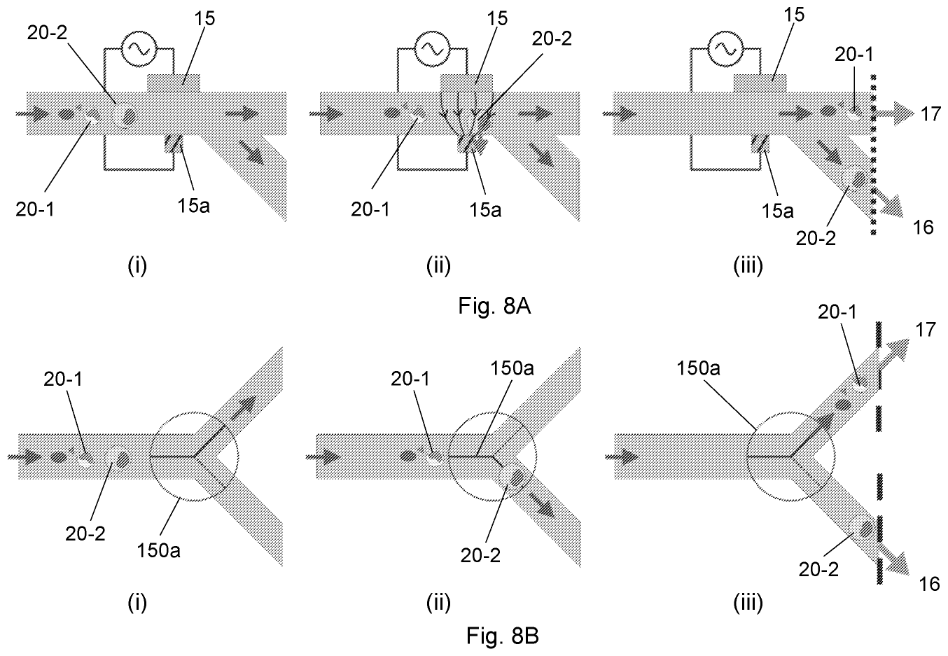

The processing unit 14 implements machine/deep learning algorithms for dealing with the large amount of data that is collected by various measurement channels. The readings gathered from the sensors are processed and a decision is made to steer tumour cells to collection reservoirs downstream of detection zone using on/off valves (dielectrophoretic, magnetic, pinched, etc.). FIGS. 8a and 8b illustrate example operations of a dielectrophoretic valve and a two-way valve, respectively.

For the dielectrophoretic valve (FIG. 8a), the liquid flow is continuous towards the collection reservoirs 16 and waste reservoirs 17. Cells approach the valve unit 15 in step (i), at which point the dielectrophoretic valve 15a is off. Tumour cell (analyte of the second type 20-2) passes through the valve unit 15 in step (ii), at which point the valve 15a is switched on and it diverts the tumour cell 20-2 towards the flow line that connects to the collection reservoir 16, while the liquid flow is not affected. Other cells (or analyte of the first type 20-1) pass through the valve unit 15 at step (iii), at which the valve 15a is off and they migrate towards the waste reservoir 17. This “off” state is the default state for the dielectrophoretic valve 15a and it is only activated when a signal for the collection is received from the sensing unit. As soon as the tumour cell 20-2 leaves the valve unit 15, the valve 15a is set back to its default state. The advantage of this valve unit 15 is that it can be integrated to the device, hence can be an inline unit as the electrode integration is already processed during manufacturing for the sensing unit. Therefore, there is no additional effort required for the integration of this dielectrophoretic valve unit.

For the two-way valve 150a in FIG. 8b, the liquid flow is either directed towards the waste reservoir 17 or collection reservoirs 16. Cells approach to the valve 150a in step (i), at which point the valve is at position 1 and the flow is directed towards the waste reservoir. Tumour cell (analyte of the second type 20-2) passes through the valve in step (ii), at which point the valve is at position 2 and the flow is directed towards the collection reservoir 16. Other cells (or analyte of the first type) pass through the valve at step (iii), at which the valve 150a is at position 1 and the flow is directed towards the waste reservoir 17. Position 1 is the default position for the two-way valve 150a and it is only set to position 2 when a signal for the collection is received from the sensing unit. As soon as the tumour cell leaves the valve, the valve is set back to its default position. The advantage of this type of valve is that many alternatives are commercially available and can easily be mounted to the system. This option does not increase the complexity of the device and it is easy to operate.

High-throughput requirements can be met by multiplexing the analysing units by utilizing modular size-based pre-sorting units, which will also be responsible for removal of the excessive carrier liquid. This approach will not only enable reaching the required throughput levels but also significantly increase the signal-to-noise ratio as the measurement volumes match with the size of the pre-sorted cells.

Combining these elements, the present disclosure will offer the first tumour cell analysis system that is able to purify and collect tumour cells without losing a single cell. Intact and viable tumour cells will be ready for downstream analysis. On-line enumeration, and digital and automated operation of the system are the key advantages.

1. A method for characterizing analytes in a sample fluid, the method comprising:
a) passing a sample liquid containing analytes of at least a first type and analytes of a second type along a flow path through a fluid channel;
b) analysing, in a first analysing unit including the fluid channel, the analytes of the at least first type and second type in the sample liquid, thereby obtaining first parameter values of at least one analyte characteristic associated with the analytes of the at least first type and second type,
c) storing, in a processing unit, the first parameter values of the at least one analyte characteristic;
d) altering a further analyte characteristic associated with the analyte of the first type compared to the further analyte characteristic associated with the analyte of the second type;
e) analysing, in a second analysing unit including the fluid channel, the analytes of the at least first type and second type in the sample liquid, thereby obtaining second parameter values of the at least one analyte characteristic associated with the analytes of the at least first type and second type;
f) storing, in the processing unit, the second parameter values of the at least one analyte characteristic;
g) characterizing, in the processing unit, the analytes of the first type from the analytes of the second type by comparing the second parameter values with the first parameter values.

2. The method according to claim 1, wherein step d) of altering the further analyte characteristic comprises the step of subjecting, in a device including the fluid channel, the analytes of the at least first type and second type in the sample liquid to an electric field.

3. The method according to claim 1, further comprising the step of
h) separating after step g), in a separating unit mounted to the fluid channel, the analytes of the first type from the analytes of the second type based on the characterization in the processing unit and using the altered further analyte characteristic of the analyte of the first type.

4. The method according to claim 3, wherein step h) comprises the steps of
h1) applying an electric field to the sample fluid or using a valve, and
h2) diverting the analyte of the second type from the flow path by activating the electric field or by guiding the flow to a desired outlet by changing the position of the valve.

5. The method according to claim 4, further comprising the step of collecting the analytes of the second type in a collection reservoir.

6. The method according to claim 1, wherein the step g) of characterizing of the analytes of the first type from the analytes of the second type is performed using one or more machine learning algorithms.

7. The method according to claim 1, wherein the steps b) and e) comprise electrical impedance spectroscopy (EIS), in particular multi-frequency electrical impedance spectroscopy.

8. The method according to claim 1, wherein step d) comprises the step of altering a membrane or causing a mechanical cell deformation of an analyte and wherein the further analyte characteristic is the electrical impedance response of the analyte.

9. The method according to claim 1, further comprising the step of optically sensing a sensing region of the fluid channel.

10. The method according to claim 1 for characterizing analytes comprising performing steps a-g concurrently in a plurality of different characterizing lines.

11. The method according to claim 10, further comprising, before step a), the step of sorting analytes in the sample fluid to different of the plurality of characterizing lines.

12. A system for characterizing analytes in a sample fluid, the system comprising at least one characterizing line, the characterizing line comprising:
a fluid channel defining a flow path having an inlet and an outlet and structured to allow a sample liquid containing analytes of at least a first type and analytes of a second type to pass through along the flow path as well as between the inlet and the outlet seen in the direction of the flow path,
a first analysing unit including the fluid channel and structured to analyse the analytes of the at least first type and second type in the sample liquid and structured to obtain first parameter values of at least one analyte characteristic associated with the analytes of the at least first type and second type,
an altering unit including the fluid channel downstream from the first analysing unit structured for altering a further analyte characteristic associated with the analyte of the first type compared to the further analyte characteristic associated with the analyte of the second type;
a second analysing unit including the fluid channel downstream from the microfluidic device and structured to analyse the analytes of the at least first type and second type in the sample liquid and structured to obtain second parameter values of the at least one analyte characteristic associated with the analytes of the at least first type and second type,
a processing unit structured to acquire and store the first parameter values and second parameter values from the first and second analysing unit and to characterize the analytes of the first type from the analytes of the second type by comparing the second parameter values with the first parameter values.

13. The system of claim 12, wherein the altering unit comprises an electric field generating unit, structured to subject the analytes of the at least first type and second type in the sample liquid to an electric field, thereby altering the further analyte characteristic associated with the analyte of the first type compared to the further analyte characteristic associated with the analyte of the second type.

14. The system according to claim 12, further comprising a separating unit including the fluid channel downstream from the second analysing unit, the separating unit structured to separate the analytes of the first type from the analytes of the second type using the altered further analyte characteristic of the analyte of the first type, based on characterization signals generated and output by the processing unit.

15. The system according to claim 14, further comprising a collection unit downstream of the separating unit for collecting analytes of the second type.

16. The system according to claim 14, wherein the separating unit comprises an electric field generating unit structured to apply an electric field to the sample fluid for diverting the analyte of the second type from the flow path.

17. The system according to claim 14, wherein the separating unit comprises a valve unit structured to divert the analyte of the second type from the flow path.

18. The system according to claim 12, further comprising a sorting unit including the fluid channel upstream from the first analysing unit and structured to sort the analytes of at least the first and second type on analyte size.

19. The system according to claim 12, wherein the processing unit implements one or more machine learning algorithms for characterizing the analytes of the first type from the analytes of the second type.

20. The system according to claim 12, wherein the first and second analysing unit comprise electrical impedance spectroscopy (EIS) means.

21. The system according to claim 12, wherein the altering unit is structured for altering a membrane or causing a mechanical cell deformation of an analyte and wherein the further analyte characteristic is an electrical impedance response.

22. The system according to claim 12, further comprising an optic sensing unit structured to optically sense a sensing region of the fluid channel.

23. A platform for characterizing analytes in a sample fluid, the platform comprising a plurality of systems according to claim 1, each system comprising a different characterizing line.

24. The platform according to claim 23, further comprising a sorting device disposed upstream of the plurality of the systems and structured to sort analytes in the sample fluid to different characterizing lines of the plurality of systems.
